(19) United States
(12) Patent Application Publication
(10) Pub. No.: US 2026/0212271 A1
(43) Pub. Date: Jul. 23, 2026

(54) INFORMATION PROCESSING DEVICE AND INFORMATION PROCESSING METHOD

(71) Applicant: NEC Corporation, Tokyo (JP)

(72) Inventors: Keisuke SUZUKI, Tokyo (JP); Mitsuru NOMA, Tokyo (JP); Osamu HISAMATSU, Tokyo (JP)

(73) Assignee: NEC Corporation, Tokyo (JP)

(21) Appl. No.: 19/442,397

(22) Filed: Jan. 7, 2026

(30) Foreign Application Priority Data
Jan. 23, 2025 (JP) 2025-010068

(51) Int. Cl. G06N 20/00; G16H 10/60

(52) U.S. Cl. CPC G06N 20/00; G16H 10/60

(57) ABSTRACT

An example object of the present disclosure is to provide a technique that enables the quality of the training data to be improved. An information processing device includes an extraction unit for extracting, from among biological data of a plurality of subjects, biological data of a subject in whom a predetermined abnormality has not been found, the biological data satisfying a predetermined condition related to the predetermined abnormality, and a generation unit for associating a label indicating that there is no predetermined abnormality with the extracted biological data and generating training data of an inference model for inferring the predetermined abnormality. According to the information processing device, the inference model can be optimized by machine learning using high-quality training data. Furthermore, by using this inference model, it is possible to support decision making by a doctor or the like who determines abnormality of the subject.

### INCORPORATION BY REFERENCE

This application is based upon and claims the benefit of priority from Japanese patent application No. 2025-010068, filed on January 23, 2025, the disclosure of which is incorporated herein in its entirety by reference.

### TECHNICAL FIELD

The present disclosure relates to an information processing device, an information processing method, and a generation program.

### BACKGROUND ART

A technique for estimating the presence or absence of abnormality using a machine-learned model obtained in which electrocardiogram data is learned is known. For example, Japanese Patent Application Laid-Open No. 2020-130772 describes an example in which a plurality of electrocardiograms are input as test data to a learned model in which a normal electrocardiogram and an abnormal electrocardiogram are learned to determine whether the electrocardiogram is normal or abnormal, and the determination accuracy is evaluated.

In the learning of the model as described above, generally, the electrocardiogram data of the visiting/hospitalized patient is used for both the positive example and the negative example. Therefore, electrocardiogram data of a patient who does not have a disease to be inferred by the model but has another disease can be used as a negative example. In addition, the result of diagnosis based on the electrocardiogram data is not necessarily correct. For example, a patient diagnosed as having an arrhythmia from the electrocardiogram data may be found not to have an arrhythmia by a subsequent electrophysiological test. Such a change in the diagnosis result may cause a false positive example.

As described above, there is a case where biological data that is invalid as a positive example or a negative example is used in generating the training data of the inference model for inferring a predetermined abnormality from the biological data, and low quality training data is generated. Then, if low-quality training data is used, an inference model with low inference accuracy may be generated.

### SUMMARY

The present disclosure has been made in view of the above problems, and an example object thereof is to provide a technique capable of improving quality of training data.

An information processing device according to an example aspect of the present disclosure includes: at least one memory configured to store instructions; and at least one processor configured to execute the instructions to: acquire biological data of a plurality of subjects from a medical examination database that records biological data obtained in a health examination undergone by the plurality of subjects; acquire medical information regarding the plurality of subjects from a medical information database that records medical care histories of the plurality of subjects; extract, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and associate a label indicating that there is no predetermined abnormality with the extracted electrocardiogram data and generate training data used for machine learning of an inference model for inferring the predetermined abnormality from electrocardiogram data.

An information processing device according to an example aspect of the present disclosure includes: at least one memory configured to store instructions; and at least one processor configured to execute the instructions to: acquire biological data of a plurality of subjects from a medical examination database that records biological data obtained in a health examination undergone by the plurality of subjects; acquire medical information regarding the plurality of subjects from a medical information database that records medical care histories of the plurality of subjects; extract, from among the biological data of the plurality of subjects, electrocardiogram data of a subject in whom a predetermined abnormality has been found, the electrocardiogram data satisfying a predetermined condition related to the predetermined abnormality, the predetermined condition being determined with reference to the medical information; and associate a label indicating that there is the predetermined abnormality with the extracted electrocardiogram data and generate training data used for machine learning of an inference model for inferring the predetermined abnormality from electrocardiogram data.

An information processing method according to an example aspect of the present disclosure includes: acquiring biological data of a plurality of subjects from a medical examination database that records biological data obtained in a health examination undergone by the plurality of subjects; acquiring medical information regarding the plurality of subjects from a medical information database that records medical care histories of the plurality of subjects; extracting, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and associating a label indicating that there is no predetermined abnormality with the extracted electrocardiogram data and generating training data used for machine learning of an inference model for inferring the predetermined abnormality from electrocardiogram data.

According to an example aspect of the present disclosure, an exemplary effect is obtained that a technique capable of improving quality of training data can be provided.

### BRIEF DESCRIPTION OF DRAWINGS

The above and other aspects, features and advantages of the present disclosure will become more apparent from the following description of certain exemplary embodiments in conjunction with the accompanying drawings, in which:

FIG. 1 is a block diagram illustrating a configuration of an information processing device according to the present disclosure;

FIG. 2 is a flowchart illustrating a flow of a generation method according to the present disclosure;

FIG. 3 is a block diagram illustrating a configuration of another information processing device according to the present disclosure;

FIG. 4 is a diagram illustrating an extraction example of biological data by the information processing device illustrated in FIG. 3;

FIG. 5 is a flowchart illustrating an example of processing executed by the information processing device illustrated in FIG. 3; and

FIG. 6 is a block diagram illustrating a configuration of a computer that functions as the information processing device according to the present disclosure.

### EXAMPLE EMBODIMENTS

Hereinafter, example embodiments of the present disclosure will be described. However, the present disclosure is not limited to the following exemplary example embodiments, and various modifications can be made within a scope described in the claims. For example, example embodiments obtained by appropriately combining techniques (some or all of things or methods) adopted in the following exemplary example embodiments can also be included in the scope of the present disclosure. Example embodiments obtained by appropriately omitting some of the techniques adopted in the following exemplary example embodiments can also be included in the scope of the present disclosure. Effects mentioned in the following exemplary example embodiments are examples of effects expected in the exemplary example embodiments, and do not define extension of the present disclosure. That is, example embodiments that do not achieve the effects mentioned in the following exemplary example embodiments can also be included in the scope of the present disclosure.

### First Exemplary Example Embodiment

A first exemplary example embodiment that is an example of the example embodiments of the present disclosure will be described in detail with reference to the drawings. The present exemplary example embodiment is a basic form of each exemplary example embodiment to be described below. An application range of each technique adopted in the present exemplary example embodiment is not limited to the present exemplary example embodiment. That is, each technique adopted in the present exemplary example embodiment can also be adopted in another exemplary example embodiment included in the present disclosure within a range in which no particular technical problem occurs. Each technique illustrated in the drawings referred to for describing the present exemplary example embodiment may also be adopted in another exemplary example embodiment included in the present disclosure within a range in which no particular technical problem occurs.

### Configuration of Information Processing Device 1

A configuration of an information processing device 1 according to the present exemplary example embodiment will be described with reference to FIG. 1. FIG. 1 is a block diagram illustrating the configuration of the information processing device 1. As illustrated in FIG. 1, the information processing device 1 includes an extraction unit 101 and a generation unit 102.

The extraction unit 101 extracts, from the biological data of a plurality of subjects, biological data of a subject in whom a predetermined abnormality has not been found, the biological data satisfying a predetermined condition related to the predetermined abnormality.

Here, the “biological data” is data derived from the subject in whom a physiological state or a state related to health/disease of the subject is reflected. The biological data may be any data that can be used to estimate whether the subject has a predetermined abnormality. For example, in a case where it is estimated whether the subject has an arrhythmia, biological data (e.g., data indicating a heart rate, electrocardiogram data, etc.) correlated with the arrhythmia may be used. The biological data may be numerical data, image data, voice data, or the like.

In addition, the “subject” is a person who is a target for acquiring biological data. In addition, the “predetermined abnormality” is an abnormality to be estimated by the inference model described below. For example, if the inference model is a model that estimates the presence or absence of the arrhythmia, the predetermined abnormality becomes the arrhythmia. Various diseases may be a predetermined abnormality, or a state that cannot be said as a disease but can be distinguished from a normal state may be a predetermined abnormality. In addition, “a predetermined abnormality has not been found” means that at least a diagnosis that there is a predetermined abnormality is not made. For example, in a case where it is diagnosed that there is no predetermined abnormality in the test using the target biological data, it may be determined that the predetermined abnormality has not been found, in a case where it is diagnosed that there is no predetermined abnormality in another test, it may be determined that the predetermined abnormality is not found, and in a case where it is diagnosed that a predetermined abnormality has not been found in a plurality of tests, it may be determined that the predetermined abnormality has not been found. The diagnosis may be performed by medical personnel such as a doctor, or may be automatic diagnosis made by a computer. Furthermore, the “predetermined condition” is a condition related to a predetermined abnormality, and may be any condition that can improve the quality of the training data by extracting the biological data that satisfy the condition. The predetermined condition will be described with an example in the second exemplary example embodiment described later.

The generation unit 102 associates the biological data extracted by the extraction unit 101 with a label indicating that there is no predetermined abnormality described above, and generates training data used for machine learning of an inference model for inferring the predetermined abnormality from the biological data. That is, the generation unit 102 generates the training data using the biological data extracted by the extraction unit 101 as a negative example.

The “inference model” may be a model that uses the biological data as input data and outputs an inference result regarding the predetermined abnormality. For example, the inference model may be such that in a case where biological data of a certain person is input, outputs a numerical value indicating a degree of possibility that the person has a predetermined abnormality. Furthermore, for example, the inference model may be a classification model. In this case, the inference model is caused to perform machine learning on the biological data related to each of the plurality of classes including the predetermined abnormality. As a result, an inference model that outputs a numerical value indicating the degree of possibility of being related to each class can be generated. The machine learning algorithm is arbitrary. For example, the inference model may be a model of a neural network system such as a Deep Neural Network (DNN) model, or may be a tree model such as a decision tree model, a support vector machine, or the like.

As described above, the information processing device 1 according to the present exemplary example embodiment adopts a configuration including the extraction unit 101 for extracting, from among biological data of a plurality of subjects, biological data of a subject in whom a predetermined abnormality has not been found, the biological data satisfying a predetermined condition related to the predetermined abnormality, and the generation unit 102 for associating a label indicating that there is no predetermined abnormality with the biological data extracted by the extraction unit 101, and generating training data used for machine learning of an inference model for inferring the predetermined abnormality from the biological data.

According to the above configuration, the biological data satisfying the predetermined condition related to the predetermined abnormality is extracted in addition to the fact that the predetermined abnormality has not been found in the subject. As a result, as compared with a case where the biological data extracted based only on the fact that the predetermined abnormality has not been found in the subject is used as the training data of the negative example, the possibility that the biological data that is not valid as the negative example is used as the training data of the negative example can be reduced. Therefore, according to the information processing device 1, an effect is obtained that the quality of the training data can be improved. Furthermore, according to the information processing device 1, the inference model can be optimized by machine learning using high-quality training data. By using this inference model, it is possible to support decision making by a doctor or the like who determines abnormality of the subject.

### Generation Program

The functions of the information processing device 1 described above can also be achieved by a program. A generation program according to the present exemplary example embodiment causes a computer to function as an extraction means for extracting, from biological data of a plurality of subjects, biological data of a subject in whom a predetermined abnormality has not been found, the biological data satisfying a predetermined condition related to the predetermined abnormality, and a generation means for associating a label indicating that there is no predetermined abnormality with the biological data extracted by the extraction means, and generating training data used for machine learning of an inference model for inferring the predetermined abnormality from the biological data. According to this generation program, an effect is obtained that the quality of the training data can be improved.

### Flow of Generation Method

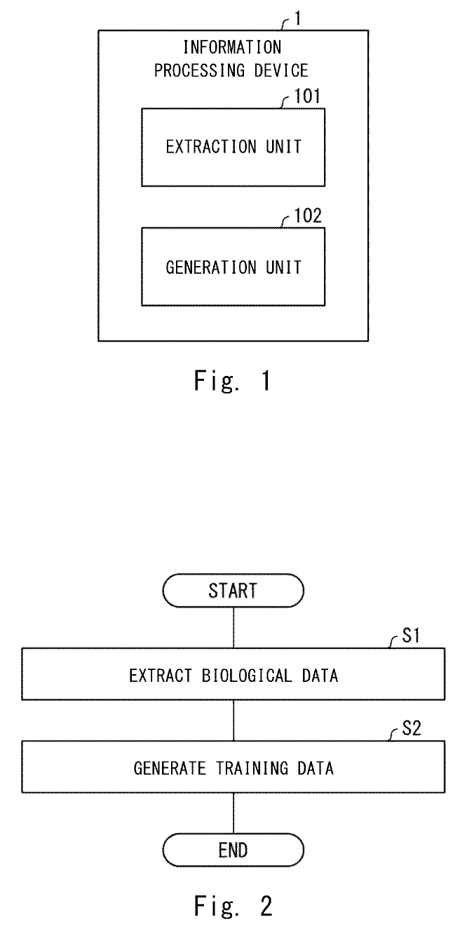

A flow of a generation method according to the present exemplary example embodiment will be described with reference to FIG. 2. FIG. 2 is a flowchart illustrating the flow of the generation method. An executing entity of each step in this generation method may be a processor included in the information processing device 1, may be a processor included in another device, or an executing entity of each step may be a processor provided in each of different devices. The generation method is also referred to herein as “information processing method”.

In S1 (extraction processing), at least one processor extracts, from the biological data of the plurality of subjects, biological data of a subject in whom a predetermined abnormality has not been found, the biological data satisfying a predetermined condition related to the predetermined abnormality.

In S2 (training data generation processing), at least one processor associates the biological data extracted in S1 with a label indicating that there is no predetermined abnormality, and generates training data used for machine learning of an inference model for inferring the predetermined abnormality from the biological data.

As described above, in the generation method according to the present exemplary example embodiment, a configuration is adopted in which at least one processor executes extraction processing of extracting, from biological data of a plurality of subjects, biological data of a subject in whom a predetermined abnormality has not been found, the biological data satisfying a predetermined condition related to the predetermined abnormality, and training data generation processing of associating a label indicating that there is no predetermined abnormality with the extracted biological data and generating training data used for machine learning of an inference model for inferring the predetermined abnormality from the biological data. According to this generation method, an effect is obtained that the quality of the training data can be improved.

### Regarding Generation of Training Data of Positive Example

The extraction unit 101 may extract, from the biological data of a plurality of subjects, biological data of a subject in whom a predetermined abnormality has been found, the biological data satisfying a predetermined condition related to the predetermined abnormality. It is similar to the case of generating the training data of the negative example in that the “predetermined condition” merely needs to be a condition related to a predetermined abnormality and capable of improving the quality of the training data by extracting the biological data satisfying the condition. The predetermined condition in the case of generating the training data of the positive example is a condition independent of the predetermined condition in the case of generating the training data of the negative example, and may be individually set. In addition, “a predetermined abnormality has been found” means that a diagnosis that there is a predetermined abnormality is made. For example, in a case where it has been diagnosed that there is a predetermined abnormality in a test using target biological data, it may be assumed that the predetermined abnormality has been found, in a case where it has been diagnosed that there is a predetermined abnormality in another test, it may be assumed that the predetermined abnormality has been found, and in a case where it has been diagnosed that there is a predetermined abnormality in a plurality of tests, it may be assumed that the predetermined abnormality has been found.

In a case where the biological data as described above is extracted, the generation unit 102 may associate the extracted biological data with a label indicating that there is predetermined abnormality, and generate training data used for machine learning of an inference model for inferring the predetermined abnormality from the biological data. In this case, the generation unit 102 generates the training data using the biological data extracted by the extraction unit 101 as a positive example.

As described above, the information processing device 1 according to the present exemplary example embodiment may include the extraction unit 101 for extracting, from among biological data of a plurality of subjects, biological data of a subject in whom a predetermined abnormality has been found, the biological data satisfying a predetermined condition related to the predetermined abnormality, and the generation unit 102 for associating a label indicating that there is predetermined abnormality with the biological data extracted by the extraction unit 101, and generating training data used for machine learning of an inference model for inferring the predetermined abnormality from the biological data.

According to the above configuration, the biological data satisfying the predetermined condition related to the predetermined abnormality is extracted in addition to the fact that the predetermined abnormality has been found in the subject. As a result, it is possible to reduce the possibility of the biological data that is not valid as the positive example being used as the training data of the negative example, as compared with the case where the biological data extracted only based on the diagnosis that there is an abnormality is used as the training data of the positive example. Therefore, according to the information processing device 1, an effect is obtained that the quality of the training data can be improved.

### Generation Program of Training Data of Positive Example

The functions of the information processing device 1 described above can also be achieved by a program. A generation program according to the present exemplary example embodiment causes a computer to function as an extraction means for extracting, from biological data of a plurality of subjects, biological data of a subject in whom a predetermined abnormality has been found, the biological data satisfying a predetermined condition related to the predetermined abnormality, and a generation means for associating a label indicating that there is predetermined abnormality with the biological data extracted by the extraction means, and generating training data used for machine learning of an inference model for inferring the predetermined abnormality from the biological data. According to this generation program, an effect is obtained that the quality of the training data can be improved.

### Generation Method of Training Data of Positive Example

The flow of the generation method in the case of generating the training data of the positive example is similar to the flow of the generation method in the case of generating the training data of the negative example described with reference to FIG. 2. That is, in a generation method in the case of generating the training data of the positive example, at least one processor executes extraction processing of extracting, from biological data of a plurality of subjects, biological data of a subject in whom a predetermined abnormality has been found, the biological data satisfying a predetermined condition related to the predetermined abnormality, and training data generation processing of associating a label indicating that there is a predetermined abnormality with the biological data extracted in the extraction processing and generating training data used for machine learning of an inference model for inferring the predetermined abnormality from the biological data. According to this generation method, an effect is obtained that the quality of the training data can be improved.

### Second Exemplary Example Embodiment

A second exemplary example embodiment that is an example of the example embodiments of the present disclosure will be described in detail with reference to the drawings. Components having the same functions as the components described in the above-described exemplary example embodiment are denoted by the same reference signs, and the description thereof will be appropriately omitted. An application range of each technique adopted in the present exemplary example embodiment is not limited to the present exemplary example embodiment. That is, each technique adopted in the present exemplary example embodiment can also be adopted in another exemplary example embodiment included in the present disclosure within a range in which no particular technical problem occurs. Each technique illustrated in each of the drawings referred to for describing the present exemplary example embodiment can be adopted in the other exemplary example embodiments included in the present disclosure within a range in which no particular technical problem occurs.

### Configuration of Information Processing Device 1A

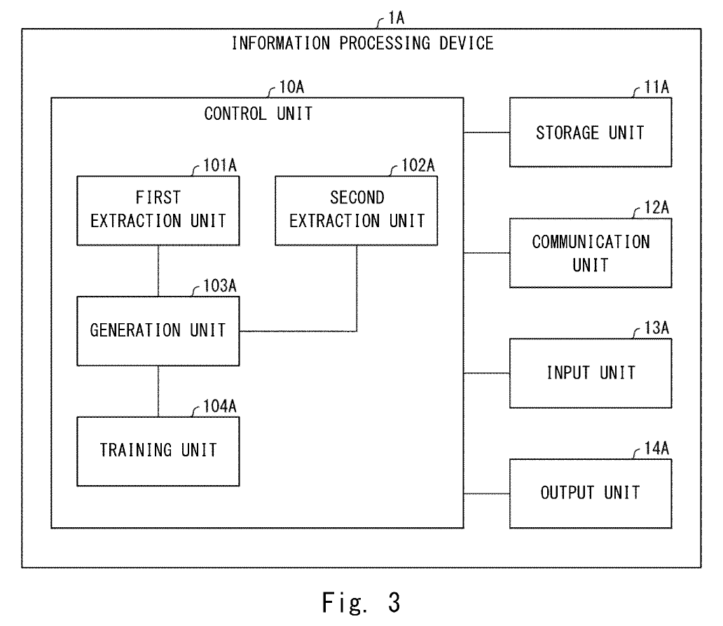

A configuration of an information processing device 1A according to the present exemplary example embodiment will be described with reference to FIG. 3. FIG. 3 is a block diagram illustrating a configuration of the information processing device 1A. The information processing device 1A is a device having a function of generating training data of an inference model and a function of generating an inference model using the generated training data.

As illustrated, the information processing device 1A includes a control unit 10A for integrally controlling each unit of the information processing device 1A, and a storage unit 11A for storing various types of data to be used by the information processing device 1A. The information processing device 1A includes a communication unit 12A for the information processing device 1A to communicate with another device, an input unit 13A for accepting an input to the information processing device 1A, and an output unit 14A for the information processing device 1A to output data. The control unit 10A includes a first extraction unit 101A, a second extraction unit 102A, a generation unit 103A, and a learning unit 104A.

Similarly to the extraction unit 101 of the first exemplary example embodiment, the first extraction unit 101A extracts, from the biological data of a plurality of subjects, biological data of a subject in whom a predetermined abnormality has not been found, the biological data satisfying a predetermined condition related to the predetermined abnormality. The biological data extracted by the first extraction unit 101A is used as a negative example.

The second extraction unit 102A extracts, from the biological data of a plurality of subjects, biological data of a subject in whom a predetermined abnormality has been found, the biological data satisfying a predetermined condition related to the predetermined abnormality. The biological data extracted by the second extraction unit 102A is used as a positive example.

Similarly to the generation unit 102 of the first exemplary example embodiment, the generation unit 103A associates the biological data extracted by the first extraction unit 101A with a label indicating that there is no predetermined abnormality, and generates training data used for machine learning of an inference model for inferring the predetermined abnormality from the biological data. The generation unit 103A associates a label indicating that there is a predetermined abnormality with the biological data extracted by the second extraction unit 102A, and generates training data used for machine learning of an inference model for inferring the predetermined abnormality from the biological data. Each of a generation unit for generating training data from the biological data extracted by the first extraction unit 101A and a generation unit for generating training data from the biological data extracted by the second extraction unit 102A may be provided.

The learning unit 104A performs machine learning using the training data generated by the generation unit 103A to generate an inference model. As in the first exemplary example embodiment, the machine learning algorithm is arbitrary.

### Regarding Predetermined Condition In Extracting Biological Data As Negative Example

The predetermined condition in a case of extracting the biological data as a negative example may include, for example, at least any of (1) the subject has no medical history of a predetermined disease related to the predetermined abnormality, (2) no congenital abnormality related to the predetermined abnormality has been found in the subject, (3) the measurer of the biological data has not found an abnormality, (4) no predetermined abnormality has been found in any of the plurality of diagnoses, (5) a blood relative of the subject has no medical history of a predetermined disease related to the predetermined abnormality, and (6) the subject has no subjective symptom related to the predetermined abnormality.

By including at least one of the above (1) to (6) in the predetermined condition in a case of extracting the biological data as the negative example, it is possible to reduce the possibility that the biological data that is not valid as the negative example is used as the training data of the negative example. Each condition will be described below.

With respect to the condition (1), although the predetermined abnormality itself has not been found, there is a possibility that the biological data of the subject having a medical history of a predetermined disease related to the predetermined abnormality may exhibit characteristics different from those of the biological data of the subject having no such medical history. The same applies to the above conditions (2) and (5), and there is a possibility that different characteristics may appear in the biological data of the subject having a congenital abnormality related to a predetermined abnormality and the biological data of the subject whose blood relative have a medical history of a predetermined disease related to the predetermined abnormality.

Since such biological data has a possibility of not being valid as a negative example, the possibility that biological data that is not valid as a negative example is used as training data of a negative example can be reduced by applying at least any one of the conditions (1), (2), and (5) described above.

Which disease is to be defined as a “predetermined disease related to a predetermined abnormality” and which abnormality is to be defined as a “congenital abnormality related to a predetermined abnormality” may be determined in advance in accordance with at least one of the biological data and the predetermined abnormality. For example, in a case where the biological data is electrocardiogram data and the predetermined abnormality is an abnormality related to the heart, the condition (1) may be that the subject has no medical history of a circulatory system disease. This is because the circulatory system disease has a possibility of affecting the electrocardiogram data. As a result, electrocardiogram data that is not affected by the circulatory system disease can be used as a negative example.

With respect to the condition (3), the measurer of the biological data often has measurement experience of the biological data for a large number of subjects, and there is a case where an abnormality that cannot be found by the test based on the biological data can be found from the experience. Therefore, by applying the condition (3), it is possible to extract highly valid biological data by utilizing the experience of the measurer. For example, in a case where the biological data is electrocardiogram data, if a determination result of the presence or absence of abnormality by medical personnel such as a technician or a nurse who has measured the electrocardiogram is recorded, the first extraction unit 101A can determine whether the condition (3) is satisfied based on the record.

With respect to the condition (4), there is a higher possibility that the subject in whom the predetermined abnormality has not been found in any of the plurality of diagnoses does not have the predetermined abnormality than the subject in whom the predetermined abnormality has not been found in one diagnosis. Therefore, by applying the condition (4), it is possible to reduce the possibility that the biological data that is not valid as the negative example is used as the training data of the negative example.

The plurality of diagnoses may be diagnosis by the same diagnosis method or diagnosis by different diagnosis methods. In the former case, for example, the first extraction unit 101A may determine that the condition (4) is satisfied in a case where a predetermined abnormality has never been found in the past diagnosis in a periodical health examination. In addition, in the latter case, for example, a plurality of diagnosis results may be obtained by using different devices for measuring biological data in each diagnosis. For example, in a case where the subject measures biological data with a wearable device, the first extraction unit 101A may determine whether the condition (4) is satisfied using a result of diagnosis using the biological data and a result of diagnosis using the biological data measured by medical personnel in a health examination or the like. These diagnoses are not limited to diagnoses by doctors and the like, and may be automatic diagnoses by devices.

With respect to the condition (6), even in a case where a minor abnormality to an extent it is not detected by the test occurs, the subject may feel the abnormality. Therefore, by applying the condition (6), it is possible to reduce the possibility that the biological data that is not valid as the negative example is used as the training data of the negative example.

### Regarding Predetermined Condition In Extracting Biological Data As Positive Example

The predetermined condition in a case of extracting biological data as a positive example may include, for example, at least any of (1) a predetermined abnormality has been found in a plurality of diagnoses, (2) it is biological data measured at the time of non-seizure of a subject having a predetermined paroxysmal abnormality, (3) no abnormality related to the predetermined abnormality is found in a diagnosis using the biological data, and (4) it is biological data obtained in a predetermined period before the treatment of the subject who has been treated for the predetermined abnormality.

By including at least one of the above (1) to (4) in the predetermined condition in a case of extracting the biological data as the positive example, it is possible to reduce the possibility that the biological data that is not valid as the positive example is used as the training data of the positive example. Each condition will be described below.

Regarding condition (1), there is a higher possibility that a subject who has been found a predetermined abnormality in a plurality of diagnoses has a predetermined abnormality than a subject who has been found a predetermined abnormality in one diagnosis. Therefore, by applying the condition (1), it is possible to reduce the possibility that the biological data that is not valid as the positive example is used as the training data of the negative example.

In addition to or in place of the condition (1), a condition that a predetermined abnormality has been found in the detailed examination may be applied. For example, in a case where an abnormality is found in an electrocardiography, an electrophysiologic test may be performed as a detailed examination. The electrophysiologic test has higher examination accuracy than electrocardiography. Therefore, in a case where the biological data is electrocardiogram data, the second extraction unit 102A may extract the electrocardiogram data on condition that a predetermined abnormality has been found in the electrophysiologic test. As a result, the possibility that electrocardiogram data that is not valid as the positive example is used as the training data of the positive example can be reduced.

Regarding condition (2), in a case where the predetermined abnormality is a paroxysmal abnormality, there is a need to diagnose the presence or absence of the predetermined abnormality from biological data measured in a state where no seizure has occurred. By applying the above condition (2), it is possible to extract biological data suitable as training data for generating an inference model according to such needs.

For example, assume that an inference model for inferring the presence or absence of paroxysmal atrial fibrillation (AF), that is, paroxysmal atrial fibrillation from the electrocardiogram data is generated. In this case, if the electrocardiogram data at the time of a seizure is used as training data of a positive example, even if the electrocardiogram data is of a patient with paroxysmal AF, the inference result that paroxysmal AF is present cannot be obtained unless the electrocardiogram data is measured at the time of a seizure. On the other hand, if the electrocardiogram data of the patient with paroxysmal AF at the time of no-seizure is used as the training data of a positive example, an inference model capable of inferring the presence or absence of paroxysmal AF from the electrocardiogram data at the time of no-seizure period can be generated.

Regarding condition (3), in a case where the biological data in which the characteristic of the abnormality related to the predetermined abnormality appears is used as the training data of the inference model for inferring the predetermined abnormality, there is a possibility that the characteristic different from the predetermined abnormality is learned as the characteristic of the predetermined abnormality and the inference accuracy of the inference model is lowered. Therefore, the possibility of decreasing the inference accuracy of the inference model can be reduced by applying the above condition (3).

For example, assume that the biological data is electrocardiogram data and the predetermined abnormality is atrial fibrillation. In this case, the predetermined condition may include that in the diagnosis using the electrocardiogram data, it is the electrocardiogram data diagnosed as not relating to any of atrial flutter, atrial premature contraction, and ventricular premature contraction. As a result, it is possible to prevent the characteristics of atrial flutter, atrial premature contraction, or ventricular premature contraction from being learned as the characteristics of atrial fibrillation, and lowering the inference accuracy of the inference model. In a case where the biological data is the electrocardiogram data, the conditions (2) and (3) may be integrated, and the fact that the biological data is the electrocardiogram data of the sinus rhythm (a state in which the sinus node that is a pacemaker of the heart is normally functioning) may be set as the predetermined condition.

Regarding condition (4), it can be said that the subject who has been treated for the predetermined abnormality had the predetermined abnormality before the treatment. Therefore, the biological data of the subject having the predetermined abnormality can be used as the training data of the positive example by applying the above condition (4). The predetermined period may be defined in advance according to a general duration of the predetermined abnormality or the like. For example, in a case where the biological data is electrocardiogram data and the predetermined abnormality is paroxysmal AF, the second extraction unit 102A may extract, for example, electrocardiogram data obtained in a period within 90 days from the treatment date as a positive example.

The extraction condition of the biological data to be the positive example/negative example is not limited to each example described above. For example, conditions such as the age of the subject at the time of measuring the biological data being within a predetermined range, the measurement date of the biological data being within a predetermined period, and the biological data being measured by applying a predetermined order type may be included in the extraction conditions. For example, in a case where the biological data is electrocardiogram data, the extraction condition may include that the order type is a standard 12 lead electrocardiogram.

### Extraction Example of Biological Data by Information Processing Device 1A

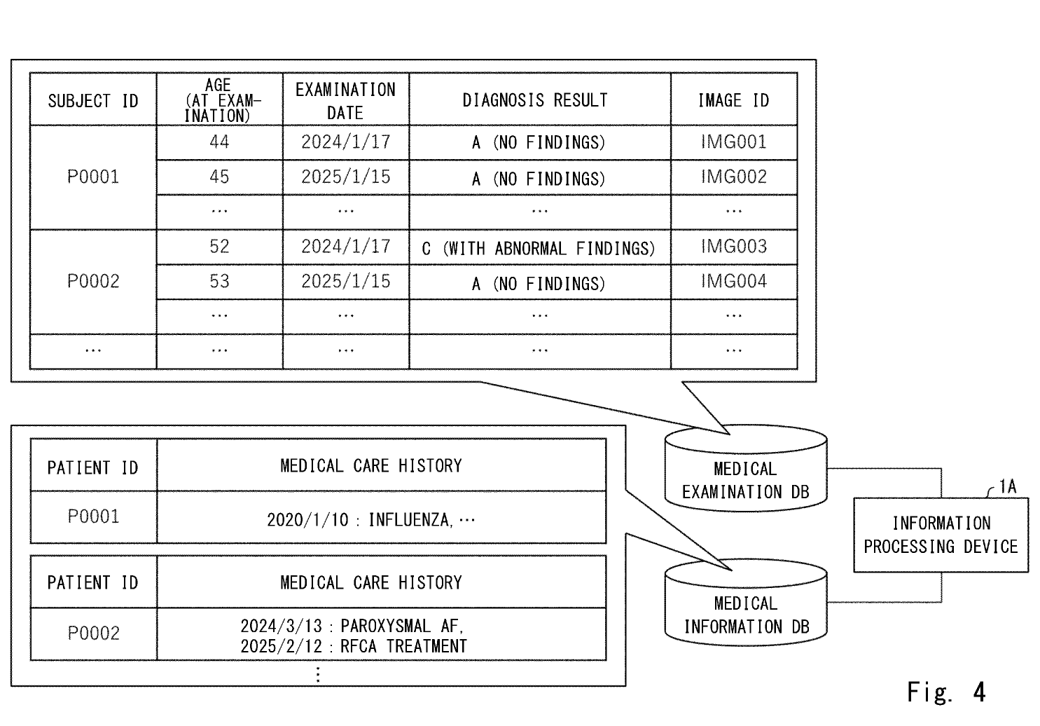

FIG. 4 is a diagram illustrating an extraction example of biological data by the information processing device 1A. More specifically, FIG. 4 illustrates an example in which electrocardiogram data as a positive example and electrocardiogram data as a negative example are extracted from the electrocardiogram data using the electrocardiogram data of a plurality of subjects recorded in a medical examination database (DB) as biological data. These pieces of electrocardiogram data are data measured by medical examination, that is, a health examination (e.g., periodic health examination). The predetermined abnormality is paroxysmal AF. “Electrocardiogram data” and “paroxysmal AF” in the following description can each be read as arbitrary “biological data” and arbitrary “predetermined abnormality”.

In the medical examination DB illustrated in FIG. 4, data in a table format in which a subject identification (ID), that is identification information of a subject, is associated with an age of the subject at the time of undergoing an electrocardiography, an examination date of the electrocardiography, a result of the electrocardiography, and an image ID, that is identification information of electrocardiogram data used for the electrocardiography, is recorded. The electrocardiogram data in this example is assumed to be image data indicating the waveform of the electrocardiogram. However, the electrocardiogram data is not limited to the image data, and may be time-series numerical data indicating cardiac potential. In addition, a combination of the image data and the numerical data may be used as the electrocardiogram data.

In addition, FIG. 4 illustrates a database called a medical information DB. The medical information DB is a database in which a medical care history in a medical institution is recorded. In the medical information DB illustrated in FIG. 4, a patient ID that is identification information of a patient and a medical care history of the patient are recorded in association with each other for each of a plurality of patients. These pieces of information may be, for example, electronic medical record. The first extraction unit 101A and the second extraction unit 102A can extract the electrocardiogram data satisfying the predetermined condition from the medical examination DB by referring to the medical information DB.

Here, in the example of FIG. 4, the subject ID in the medical examination DB and the patient ID in the medical information DB are common. That is, a person whose subject ID and patient ID are the same is the same person. For example, a person whose subject ID is “P0001” in the medical examination DB of FIG. 4 is the same person as a person whose patient ID is “P0001” in the medical information DB. In this manner, the satisfaction determination of the condition across the medical examination DB and the medical information DB can be accurately and rapidly performed by making the identification information of the subject/patient common between the medical examination DB and the medical information DB. Although only one medical information DB and one medical examination DB are illustrated in FIG. 4, satisfaction determination of a condition and extraction of electrocardiogram data may be performed with reference to a plurality of medical information DBs and medical examination DBs. In this case as well, it is preferable to have the identification information of the subject/patient made common in each DB. For example, an insurance identification number, a personal identity number, or the like may be used as the common identification information.

The first extraction unit 101A may determine, for each piece of electrocardiogram data recorded in the medical examination DB, whether the electrocardiogram data is the electrocardiogram data of the subject in whom a predetermined abnormality (specifically, paroxysmal AF) has been found and whether the electrocardiogram data satisfies a predetermined condition related to paroxysmal AF. Then, the first extraction unit 101A may extract, from the medical examination DB, the electrocardiogram data determined to be the electrocardiogram data of the subject in whom paroxysmal AF has not been found and the electrocardiogram data satisfying a predetermined condition related to paroxysmal AF.

For example, in the medical examination DB illustrated in FIG. 4, in the electrocardiogram data having the image ID “IMG001”, the diagnosis result is “A (no findings)”. In addition, in the medical care history of the subject (subject with the subject ID “P0001”) corresponding to this electrocardiogram data, a disease called influenza is indicated, but a circulatory system disease is not indicated. Therefore, the first extraction unit 101A determines that the electrocardiogram data with the image ID “IMG001” is the electrocardiogram data of the subject in whom paroxysmal AF has not been found and the electrocardiogram data satisfying the predetermined condition related to paroxysmal AF, and extracts the electrocardiogram data. In a case where a classification symbol according to International Classification of Diseases (ICD) is described in the medical care history, the presence or absence of a circulatory system disease can be determined based on the classification symbol. For example, in a case where the classification symbol of ICD-10 is used in the medical care history, the first extraction unit 101A may determine a disease having a classification symbol starting with “I” as a circulatory system disease.

On the other hand, the electrocardiogram data with the image ID “IMG004” also has the diagnosis result “A (no findings)” similarly to the electrocardiogram data with the image ID “IMG001”. However, the medical care history of the subject (subject with the subject ID “P0002”) corresponding to this electrocardiogram data shows a medical history of paroxysmal AF. Therefore, the first extraction unit 101A determines that the electrocardiogram data with the image ID “IMG004” does not satisfy the condition that the electrocardiogram data is the electrocardiogram data of the subject in whom the predetermined abnormality (specifically, paroxysmal AF) has not been found, and excludes the electrocardiogram data from the target of extraction.

Similarly, the second extraction unit 102A may determine, for each piece of electrocardiogram data recorded in the medical examination DB, whether the electrocardiogram data is the electrocardiogram data of the subject in whom paroxysmal AF has been found and whether the electrocardiogram data satisfies a predetermined condition related to paroxysmal AF. Then, the second extraction unit 102A may extract, from the medical examination DB, the electrocardiogram data of the subject in whom paroxysmal AF has been found and determined to satisfy a predetermined condition related to paroxysmal AF.

For example, in the medical examination DB illustrated in FIG. 4, in the electrocardiogram data with the image ID “IMG004”, the diagnosis result is “A (no findings)”. However, the medical care history of the subject (subject with the subject ID “P0002”) corresponding to this electrocardiogram data shows a medical history of paroxysmal AF. Here, it is assumed that the predetermined condition at the time of extracting the biological data as a positive example is the electrocardiogram data obtained in the period of 90 days before the treatment date of the subject who has been treated for paroxysmal AF. In this case, the second extraction unit 102A refers to the medical information DB and specifies the date on which the RFCA (radiofrequency catheter ablation) treatment, which is treatment for paroxysmal AF, is performed, that is, the treatment date. Then, the second extraction unit 102A refers to the medical examination DB and determines whether the electrocardiogram data of “IMG004” is data obtained in a period of 90 days before the treatment date. In the example of FIG. 4, since the date on which the electrocardiogram data of “IMG004” is measured (that is, the examination date) is January 15 in 2025 and the treatment date is February 12 in 2025, it is determined that the predetermined condition is satisfied. Therefore, the second extraction unit 102A extracts the electrocardiogram data with the image ID “IMG004”.

On the other hand, the electrocardiogram data with the image ID “IMG003 ” is also the electrocardiogram data of the subject (patient with the patient ID “P0002”) in whom paroxysmal AF has been found, similarly to the electrocardiogram data with “IMG004”. Here, the result of the diagnosis based on the electrocardiogram data is “C (with abnormal findings)”. Therefore, in a case where the predetermined condition includes the condition that (2) paroxysmal AF is not found in the diagnosis using the biological data, the second extraction unit 102A excludes the electrocardiogram data with the image ID “IMG003” from the target of extraction.

As described above, the first extraction unit 101A and the second extraction unit 102A may extract the biological data from the medical examination DB that records the biological data obtained in the health examination undergone by a plurality of subjects, and determine whether the predetermined condition is satisfied with reference to the medical information DB that records the medical care histories of the plurality of subjects. This makes it possible to extract valid biological data from the medical examination DB by utilizing the medical information DB.

As described above, the first extraction unit 101A and the second extraction unit 102A may extract biological data associated with a subject ID that is identification information of a subject in the medical examination DB, and determine whether a predetermined condition is satisfied with reference to a medical care history associated with the ID among medical care histories recorded in the medical information DB. As a result, processing of extracting valid biological data with reference to a plurality of different databases such as the medical examination DB and the medical information DB can be smoothly performed. From which database the biological data is extracted is arbitrary. For example, part of the biological data may be extracted from the medical examination DB, and the other part may be acquired from the medical information DB. In particular, in a case where the biological data obtained in a predetermined period before the treatment of the subject who has been treated for the predetermined abnormality is extracted, there is a high possibility that the biological data has been measured in the period in the hospital where the treatment has been performed (e.g., the hospital where the subject has been hospitalized). Therefore, in a case where such biological data is extracted, the biological data may be extracted with the medical information DB as the target.

### Flow of Processing

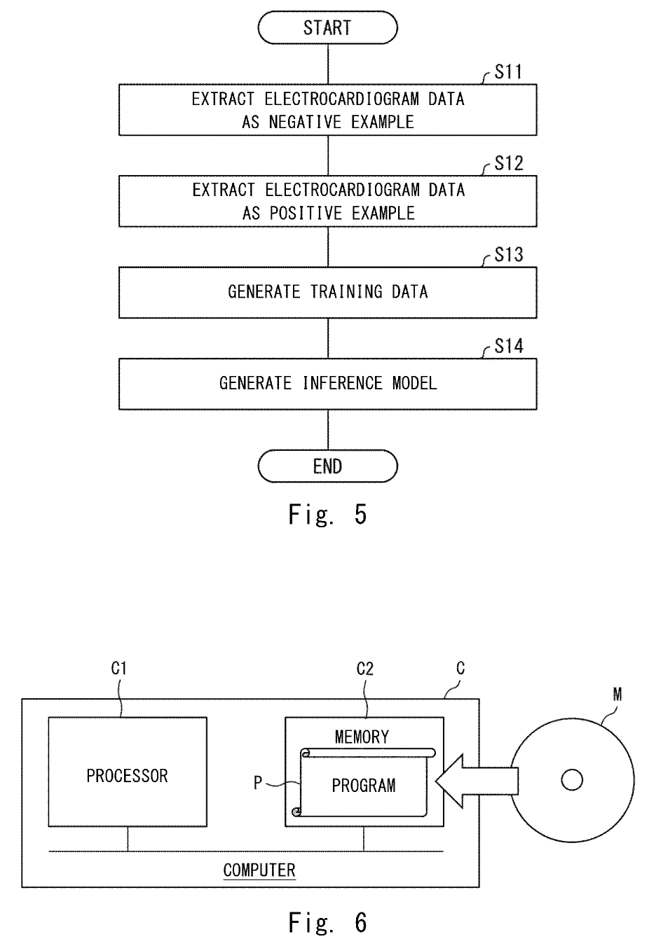

A flow of processing executed by the information processing device 1A will be described with reference to FIG. 5. FIG. 5 is a flowchart illustrating an example of processing executed by the information processing device 1A. The flowchart of FIG. 5 includes each processing of the generation method according to the present exemplary example embodiment. FIG. 5 illustrates an example in which the biological data is electrocardiogram data. In the following description, the predetermined abnormality is paroxysmal AF. In the following description, “electrocardiogram data” and “paroxysmal AF” can each be read as arbitrary “biological data” and arbitrary “predetermined abnormality”.

In S11 (extraction processing), the first extraction unit 101A extracts, from the electrocardiogram data of the plurality of subjects, the electrocardiogram data that is the biological data of the subject in whom the paroxysmal AF has not been found and that satisfies the predetermined condition related to the paroxysmal AF. For example, the first extraction unit 101A may extract the electrocardiogram data from the medical examination DB with reference to the medical examination DB and the medical information DB as illustrated in FIG. 4.

In S12 (extraction processing), the second extraction unit 102A extracts, from the electrocardiogram data of the plurality of subjects, the electrocardiogram data that is the biological data of the subject in whom the paroxysmal AF has been found and that satisfies the predetermined condition related to the paroxysmal AF. For example, the second extraction unit 102A may extract the electrocardiogram data from the medical examination DB with reference to the medical examination DB and the medical information DB as illustrated in FIG. 4. The processing of S12 may be performed before S11, or the processing may be performed in parallel.

In S13 (training data generation processing), the generation unit 103A associates the electrocardiogram data extracted in S11 with a label indicating that there is no paroxysmal AF, and generates training data used for machine learning of an inference model for inferring paroxysmal AF from the electrocardiogram data. In addition, the generation unit 103A generates training data used for machine learning of the inference model by associating a label indicating there is paroxysmal AF with the electrocardiogram data extracted in S12.

In S14 (learning processing), the learning unit 104A performs machine learning using the training data generated in S13 to generate the inference model. Accordingly, the processing of FIG. 5 ends. The inference model may be generated by another device different from the information processing device 1A. In this case, the processing (generation method of training data) executed by the information processing device 1A ends in S13. Furthermore, in this case, the information processing device 1A does not necessarily perform both S11 and S12.

As described above, the generation method according to the present exemplary example embodiment includes extraction processing of extracting, from among biological data of a plurality of subjects, biological data of a subject in whom a predetermined abnormality has not been found, the biological data satisfying a predetermined condition related to the predetermined abnormality, training data generation processing of associating a label indicating that there is no predetermined abnormality with the biological data extracted in the extraction processing and generating training data used for machine learning of an inference model for inferring the predetermined abnormality from the biological data, and model generation processing of performing machine learning using the generated training data and generating the inference model. According to this generation method, it is possible to improve the quality of the training data, thereby obtaining an effect that an inference model with high inference accuracy can be generated.

Furthermore, as described above, the generation method according to the present exemplary example embodiment includes extraction processing of extracting, from among biological data of a plurality of subjects, biological data of a subject in whom a predetermined abnormality has been found, the biological data satisfying a predetermined condition related to the predetermined abnormality, training data generation processing of associating a label indicating that there is predetermined abnormality with the biological data extracted in the extraction processing and generating training data used for machine learning of an inference model for inferring the predetermined abnormality from the biological data, and model generation processing of performing machine learning using the generated training data and generating the inference model. According to this generation method, it is possible to improve the quality of the training data, thereby obtaining an effect that an inference model with high inference accuracy can be generated.

In a case where the electrocardiogram data is the image data, in S13, the generation unit 103A may cut out a portion of the electrocardiogram data to obtain the training data. The portion to be cut out may be designated by the user of the information processing device 1A via the communication unit 12A or the input unit 13A. In addition, a tool such as Robotic Process Automation (RPA) may be used in order to save the work related to such cut-out.

Furthermore, in FIG. 5, the processing of S11 is assumed as one step, but this processing may be divided into a plurality of steps. For example, the first extraction unit 101A may first extract electrocardiogram data having a diagnosis result of “A (no findings)” from the electrocardiogram data recorded in the medical examination DB, and then determine whether each piece of extracted electrocardiogram data satisfies a predetermined condition. The same applies to S12.

### Modified Examples

An executing entity of each processing described in the above-described exemplary example embodiments is optional, and is not limited to the above-described examples. For example, a system having functions similar to those of the information processing devices 1 and 1A can be constructed by a plurality of devices capable of communicating with each other. The executing entity of each processing illustrated in the flowchart of FIG. 5 may be one device (may be rephrased as a processor) or a plurality of devices (may be similarly rephrased as processors).

### Implementation Example by Software

Some or all of the functions of the information processing devices 1 and 1A (hereinafter, also referred to as “each of the above devices”) may be implemented by hardware such as an integrated circuit (IC chip) or may be implemented by software.

In the latter case, each of the above devices is implemented by, for example, a computer that executes a command of a program as software for implementing each function. An example of such a computer (hereinafter referred to as a computer C) is illustrated in FIG. 6. FIG. 6 is a block diagram illustrating a hardware configuration of the computer C that functions as each of the above devices.

The computer C includes at least one processor C1 and at least one memory C2. A program (generation program) P for operating the computer C as each of the above devices is recorded in the memory C2. In the computer C, the processor C1 reads the program P from the memory C2 and executes the program, thereby implementing the functions of each of the above devices.

As the processor C1, for example, a Central Processing Unit (CPU), a Graphic Processing Unit (GPU), a Digital Signal Processor (DSP), a Micro Processing Unit (MPU), a Floating point number Processing Unit (FPU), a Physics Processing Unit (PPU), a Tensor Processing Unit (TPU), a quantum processor, a microcontroller, a combination thereof, or the like can be used. As the memory C2, for example, a flash memory, a Hard Disk Drive (HDD), a Solid State Drive (SSD), a combination thereof, or the like can be used.

The computer C may further include a Random Access Memory (RAM) for loading the program P at the time of execution and temporarily storing various data. The computer C may further include a communication interface for transmitting and receiving data to and from another device. The computer C may further include an input/output interface for connecting input/output equipment such as a keyboard, a mouse, a display, and a printer.

The program can be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), and semiconductor memories (such as mask ROM, PROM (programmable ROM), EPROM (erasable PROM), flash ROM, RAM (random access memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Each of the above functions of each of the above devices may be implemented by a single processor provided in a single computer, may be implemented by cooperation of a plurality of processors provided in a single computer, or may be implemented by cooperation of a plurality of processors provided in a plurality of computers. The program for causing each of the above devices to implement each of the above functions may be stored in a single memory provided in a single computer, may be stored in a distributed manner in a plurality of memories provided in a single computer, or may be stored in a distributed manner in a plurality of memories provided in a plurality of computers.

### Supplementary Information 1

The present disclosure includes the technologies described in the following Supplementary Notes. However, the present disclosure is not limited to the techniques described in the following supplementary notes, and various modifications can be made within the scope described in the claims.

### Supplementary Note A1

An information processing device including an extraction means for extracting, from among biological data of a plurality of subjects, biological data of a subject in whom a predetermined abnormality has not been found, the biological data satisfying a predetermined condition related to the predetermined abnormality, and a generation means for associating a label indicating that there is no predetermined abnormality with the biological data extracted by the extraction means and generating training data used for machine learning of an inference model for inferring the predetermined abnormality from the biological data.

### Supplementary Note A2

The information processing device according to supplementary note A1, in which the predetermined condition includes at least any of (1) the subject has no medical history of a predetermined disease related to the predetermined abnormality, (2) no congenital abnormality related to the predetermined abnormality has been found in the subject, (3) no abnormality has been found by a measurer of the biological data, (4) no predetermined abnormality has been found in any of a plurality of diagnoses, (5) a blood relative of the subject has no medical history of a predetermined disease related to the predetermined abnormality, and (6) the subject has no subjective symptom related to the predetermined abnormality.

### Supplementary Note A3

The information processing device according to supplementary note A2, in which the biological data is electrocardiogram data, and the predetermined condition includes that the subject has no medical history of a circulatory system disease.

### Supplementary Note A4

An information processing device including an extraction means for extracting, from among biological data of a plurality of subjects, biological data of a subject in whom a predetermined abnormality has been found, the biological data satisfying a predetermined condition related to the predetermined abnormality, and a generation means for generating training data to be used for machine learning of an inference model for inferring, from the biological data, a predetermined abnormality by associating a label indicating that there is the predetermined abnormality with the biological data extracted by the extraction means.

### Supplementary Note A5

The information processing device according to supplementary note A4, in which the predetermined condition includes at least any of (1) the predetermined abnormality has been found in a plurality of diagnoses, (2) the predetermined abnormality has not been found in a diagnosis using the biological data, (3) no abnormality related to the predetermined abnormality has been found in a diagnosis using the biological data, and (4) biological data is obtained in a predetermined period before treatment of the subject who has been treated for the predetermined abnormality.

### Supplementary Note A6

The information processing device according to supplementary note A5, in which the biological data is electrocardiogram data, the predetermined abnormality is atrial fibrillation, and the predetermined condition includes being electrocardiogram data diagnosed as not corresponding to any of atrial flutter, atrial premature contraction, and ventricular premature contraction in a diagnosis using the electrocardiogram data.

### Supplementary Note A7

The information processing device according to any one of supplementary notes A1 to A6, in which the extraction means extracts biological data from a medical examination database that records biological data obtained in a health examination undergone by the plurality of subjects, and determines whether the predetermined condition is satisfied with reference to a medical information database that records medical care histories of the plurality of subjects.

### Supplementary Note A8

The information processing device according to supplementary note A7, in which the extraction means determines whether biological data associated with identification information of the subject in the medical examination database satisfies the predetermined condition with reference to a medical care history associated with the identification information among medical care histories recorded in the medical information database.

### Supplementary Note B1

An information processing method including: acquiring biological data of a plurality of subjects from a medical examination database that records biological data obtained in a health examination undergone by the plurality of subjects; acquiring medical information regarding the plurality of subjects from a medical information database that records medical care histories of the plurality of subjects; extracting, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and associating a label indicating that there is no predetermined abnormality with the extracted electrocardiogram data and generating training data used for machine learning of an inference model for inferring the predetermined abnormality from electrocardiogram data.

### Supplementary Note B2

The information processing method according to supplementary note B1, in which the predetermined condition further includes that no abnormality has been found by a measurer of the electrocardiogram data.

### Supplementary Note B3

The information processing method according to supplementary note B2, in which the predetermined condition further includes that, in a plurality of diagnoses based on the electrocardiogram data of the subject, the predetermined abnormality is not determined to be present.

### Supplementary Note B4

An information processing method including: acquiring biological data of a plurality of subjects from a medical examination database that records biological data obtained in a health examination undergone by the plurality of subjects; acquiring medical information regarding the plurality of subjects from a medical information database that records medical care histories of the plurality of subjects; extracting, from among the biological data of the plurality of subjects, electrocardiogram data of a subject in whom a predetermined abnormality has been found, the electrocardiogram data satisfying a predetermined condition related to the predetermined abnormality, the predetermined condition being determined with reference to the medical information; and associating a label indicating that there is the predetermined abnormality with the extracted electrocardiogram data and generating training data used for machine learning of an inference model for inferring the predetermined abnormality from electrocardiogram data.

### Supplementary Note B5

The information processing method according to supplementary note B4, in which the predetermined condition includes that the electrocardiogram data has been obtained in a predetermined period before treatment of the subject who has been treated for the predetermined abnormality.

### Supplementary Note B6

The information processing method according to supplementary note B5, in which the predetermined abnormality is atrial fibrillation, the predetermined condition includes being electrocardiogram data diagnosed as not corresponding to any of atrial flutter, atrial premature contraction, and ventricular premature contraction in a diagnosis using the electrocardiogram data.

### Supplementary Note B7

The information processing method according to any one of supplementary notes B1 to B6, in which the at least one processor specifies, based on medical information acquired from a medical information database that records medical care histories of the plurality of subjects, a subject that satisfies the predetermined condition, and acquires electrocardiogram data of the specified subject from a medical examination database recording biological data obtained in a health examination undergone by the plurality of subjects.

### Supplementary Note B8

The information processing method according to supplementary note B7, in which the at least one processor determines whether electrocardiogram data associated with identification information of the subject in the medical examination database satisfies the predetermined condition with reference to a medical care history associated with the identification information among medical care histories recorded in the medical information database.

### Supplementary Note B9

An information processing method in which at least one processor executes extraction processing of extracting, from among biological data of a plurality of subjects, biological data of a subject in whom a predetermined abnormality has not been found, the biological data satisfying a predetermined condition related to the predetermined abnormality, training data generation processing of associating a label indicating that there is no predetermined abnormality with the biological data extracted in the extraction processing and generating training data used for machine learning of an inference model for inferring the predetermined abnormality from the biological data, and model generation processing of performing machine learning using the training data and generating the inference model.

### Supplementary Note B10

An information processing method in which at least one processor executes extraction processing of extracting, from among biological data of a plurality of subjects, biological data of a subject in whom a predetermined abnormality has been found, the biological data satisfying a predetermined condition related to the predetermined abnormality, training data generation processing of associating a label indicating that there is predetermined abnormality with the biological data extracted in the extraction processing and generating training data used for machine learning of an inference model for inferring the predetermined abnormality from the biological data, and model generation processing of performing machine learning using the training data and generating the inference model.

### Supplementary Note C1

A generation program for causing a computer to function as an extraction means for extracting, from biological data of a plurality of subjects, biological data of a subject in whom a predetermined abnormality has not been found, the biological data satisfying a predetermined condition related to the predetermined abnormality, and a generation means for associating a label indicating that there is no predetermined abnormality with the biological data extracted by the extraction means, and generating training data used for machine learning of an inference model for inferring the predetermined abnormality from the biological data.

### Supplementary Note C2

The generation program according to supplementary note C1, in which the predetermined condition includes at least any of (1) the subject has no medical history of a predetermined disease related to the predetermined abnormality, (2) no congenital abnormality related to the predetermined abnormality has been found in the subject, (3) no abnormality has been found by a measurer of the biological data, (4) no predetermined abnormality has been found in any of a plurality of diagnoses, (5) a blood relative of the subject has no medical history of a predetermined disease related to the predetermined abnormality, and (6) the subject has no subjective symptom related to the predetermined abnormality.

### Supplementary Note C3

The generation program according to supplementary note C2, in which the biological data is electrocardiogram data, and the predetermined condition includes that the subject has no medical history of a circulatory system disease.

### Supplementary Note C4

A generation program for causing a computer to function as an extraction means for extracting, from biological data of a plurality of subjects, biological data of a subject in whom a predetermined abnormality has been found, the biological data satisfying a predetermined condition related to the predetermined abnormality, and a generation means for associating a label indicating that there is predetermined abnormality with the biological data extracted by the extraction means, and generating training data used for machine learning of an inference model for inferring the predetermined abnormality from the biological data.

### Supplementary Note C5

The generation program according to supplementary note C4, in which the predetermined condition includes at least any of (1) the predetermined abnormality has been found in a plurality of diagnoses, (2) the predetermined abnormality has not been found in a diagnosis using the biological data, (3) no abnormality related to the predetermined abnormality has been found in a diagnosis using the biological data, and (4) biological data is obtained in a predetermined period before treatment of the subject who has been treated for the predetermined abnormality.

### Supplementary Note C6

The generation program according to supplementary note C5, in which the biological data is electrocardiogram data, the predetermined abnormality is atrial fibrillation, the predetermined condition includes being electrocardiogram data diagnosed as not corresponding to any of atrial flutter, atrial premature contraction, and ventricular premature contraction in a diagnosis using the electrocardiogram data.

### Supplementary Note C7

The generation program according to any one of supplementary notes C1 to C6, in which the extraction means extracts biological data from a medical examination database that records biological data obtained in a health examination undergone by the plurality of subjects, and determines whether the predetermined condition is satisfied with reference to a medical information database that records medical care histories of the plurality of subjects.

### Supplementary Note C8

The generation program according to supplementary note C7, in which the extraction means determines whether biological data associated with identification information of the subject in the medical examination database satisfies the predetermined condition with reference to a medical care history associated with the identification information among medical care histories recorded in the medical information database.

### Supplementary Note D1

An information processing device including at least one processor, the at least one processor configured to execute instructions to: acquire biological data of a plurality of subjects from a medical examination database that records biological data obtained in a health examination undergone by the plurality of subjects; acquire medical information regarding the plurality of subjects from a medical information database that records medical care histories of the plurality of subjects extract, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and associate a label indicating that there is no predetermined abnormality with the extracted electrocardiogram data and generate training data used for machine learning of an inference model for inferring the predetermined abnormality from electrocardiogram data.

The information processing device may further include a memory. The memory may store a program for causing the at least one processor to execute each of the processing.

### Supplementary Note D2

The information processing device according to supplementary note D1, in which the predetermined condition further includes that no abnormality has been found by a measurer of the electrocardiogram data.

### Supplementary Note D3

The information processing device according to supplementary note D2, in which the predetermined condition further includes that, in a plurality of diagnoses based on the electrocardiogram data of the subject, the predetermined abnormality is not determined to be present.

### Supplementary Note D4

An information processing device including at least one processor, the at least one processor configured to execute instructions to: acquire biological data of a plurality of subjects from a medical examination database that records biological data obtained in a health examination undergone by the plurality of subjects; acquire medical information regarding the plurality of subjects from a medical information database that records medical care histories of the plurality of subjects; extract, from among the biological data of the plurality of subjects, electrocardiogram data of a subject in whom a predetermined abnormality has been found, the electrocardiogram data satisfying a predetermined condition related to the predetermined abnormality, the predetermined condition being determined with reference to the medical information; and associate a label indicating that there is the predetermined abnormality with the extracted electrocardiogram data and generate training data used for machine learning of an inference model for inferring the predetermined abnormality from electrocardiogram data.

### Supplementary Note D5

The information processing device according to supplementary note D4, in which the predetermined condition includes that the electrocardiogram data has been obtained in a predetermined period before treatment of the subject who has been treated for the predetermined abnormality.

### Supplementary Note D6

The information processing device according to supplementary note D5, in which the predetermined abnormality is atrial fibrillation, and the predetermined condition includes being electrocardiogram data diagnosed as not corresponding to any of atrial flutter, atrial premature contraction, and ventricular premature contraction in a diagnosis using the electrocardiogram data.

### Supplementary Note D7

The information processing device according to any one of supplementary notes D1 to D6, in which the at least one processor specifies, based on medical information acquired from a medical information database that records medical care histories of the plurality of subjects, a subject that satisfies the predetermined condition, and acquires electrocardiogram data of the specified subject from a medical examination database recording biological data obtained in a health examination undergone by the plurality of subjects.

### Supplementary Note D8

The information processing device according to supplementary note D7, in which the at least one processor determines whether electrocardiogram data associated with identification information of the subject in the medical examination database satisfies the predetermined condition with reference to a medical care history associated with the identification information among medical care histories recorded in the medical information database.

### Supplementary Note D9

The information processing device according to supplementary note D1, in which the predetermined condition further includes that no congenital abnormality related to the predetermined abnormality has been found in the subject.

### Supplementary Note D10

The information processing device according to supplementary note D1, in which the predetermined condition further includes that a blood relative of the subject has no medical history of a predetermined disease related to the predetermined abnormality.

### Supplementary Note D11

The information processing device according to supplementary note D4, in which the predetermined condition includes that the predetermined abnormality has been found in a plurality of diagnoses for the subject.

### Supplementary Note D12

The information processing device according to supplementary note D4, in which the predetermined condition includes that the predetermined abnormality has not been found in a diagnosis using the electrocardiogram data.

### Supplementary Note D13

The information processing device according to supplementary note D4, in which the predetermined condition includes that no abnormality related to the predetermined abnormality has been found in a diagnosis using the electrocardiogram data.

### Supplementary Note E1

A non-transitory recording medium recorded with a generation program for causing a computer to function as an information processing device, the program causing the computer to execute extraction processing of extracting, from among biological data of a plurality of subjects, biological data of a subject in whom a predetermined abnormality has not been found, the biological data satisfying a predetermined condition related to the predetermined abnormality, and training data generation processing of associating a label indicating that there is no predetermined abnormality with the biological data extracted in the extraction processing and generating training data used for machine learning of an inference model for inferring the predetermined abnormality from the biological data.

### Supplementary Note E2

A non-transitory recording medium recorded with a generation program for causing a computer to execute extraction processing of extracting, from among biological data of a plurality of subjects, biological data of a subject in whom a predetermined abnormality has been found, the biological data satisfying a predetermined condition related to the predetermined abnormality, and training data generation processing of associating a label indicating that there is predetermined abnormality with the biological data extracted in the extraction processing and generating training data used for machine learning of an inference model for inferring the predetermined abnormality from the biological data.

The first and second embodiments can be combined as desirable by one of ordinary skill in the art.

While the disclosure has been particularly shown and described with reference to embodiments thereof, the disclosure is not limited to these embodiments. It will be understood by those of ordinary skill in the art that various changes in form and details may be made therein without departing from the spirit and scope of the present disclosure as defined by the claims.

What is claimed is:

1. An information processing apparatus comprising:
at least one memory configured to store instructions; and
at least one processor configured to execute the instructions to:
acquire biological data of a plurality of subjects from a medical examination database that records biological data obtained in a health examination undergone by the plurality of subjects;
acquire medical information regarding the plurality of subjects from a medical information database that records medical care histories of the plurality of subjects;
extract, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and
associate a label indicating that there is no predetermined abnormality with the extracted electrocardiogram data and generate training data used for machine learning of an inference model for inferring the predetermined abnormality from electrocardiogram data.

2. The information processing apparatus according to claim 1, wherein
the predetermined condition further includes that no abnormality has been found by a measurer of the electrocardiogram data.

3. The information processing apparatus according to claim 2, wherein
the predetermined condition further includes that, in a plurality of diagnoses based on the electrocardiogram data of the subject, the predetermined abnormality is not determined to be present.

4. An information processing apparatus comprising:
at least one memory configured to store instructions; and
at least one processor configured to execute the instructions to:
acquire biological data of a plurality of subjects from a medical examination database that records biological data obtained in a health examination undergone by the plurality of subjects;
acquire medical information regarding the plurality of subjects from a medical information database that records medical care histories of the plurality of subjects;
extract, from among the biological data of the plurality of subjects, electrocardiogram data of a subject in whom a predetermined abnormality has been found, the electrocardiogram data satisfying a predetermined condition related to the predetermined abnormality, the predetermined condition being determined with reference to the medical information; and
associate a label indicating that there is the predetermined abnormality with the extracted electrocardiogram data and generate training data used for machine learning of an inference model for inferring the predetermined abnormality from electrocardiogram data.

5. The information processing apparatus according to claim 4, wherein
the predetermined condition includes that the electrocardiogram data has been obtained in a predetermined period before treatment of the subject who has been treated for the predetermined abnormality.

6. The information processing apparatus according to claim 5, wherein
the predetermined abnormality is atrial fibrillation, and
the predetermined condition includes being electrocardiogram data diagnosed as not corresponding to any of atrial flutter, atrial premature contraction, and ventricular premature contraction in a diagnosis using the electrocardiogram data.

7. The information processing apparatus according to claim 1, wherein
the at least one processor specifies, based on medical information acquired from a medical information database that records medical care histories of the plurality of subjects, a subject that satisfies the predetermined condition, and acquires electrocardiogram data of the specified subject from a medical examination database recording biological data obtained in a health examination undergone by the plurality of subjects.

8. The information processing apparatus according to claim 7, wherein
the at least one processor determines whether electrocardiogram data associated with identification information of the subject in the medical examination database satisfies the predetermined condition with reference to a medical care history associated with the identification information among medical care histories recorded in the medical information database.

9. An information processing method comprising:
acquiring biological data of a plurality of subjects from a medical examination database that records biological data obtained in a health examination undergone by the plurality of subjects;
acquiring medical information regarding the plurality of subjects from a medical information database that records medical care histories of the plurality of subjects;
extracting, from among the biological data of the plurality of subjects, electrocardiogram data of a subject in whom a predetermined abnormality has not been found, the electrocardiogram data satisfying a predetermined condition related to the predetermined abnormality, the predetermined condition including that the subject has no medical history of a circulatory system disease and has no subjective symptom related to the predetermined abnormality, the predetermined condition being determined with reference to the medical information; and
associating a label indicating that there is no predetermined abnormality with the extracted electrocardiogram data and generating training data used for machine learning of an inference model for inferring the predetermined abnormality from electrocardiogram data.

10. The information processing method according to claim 9, wherein
the predetermined condition further includes that no abnormality has been found by a measurer of the electrocardiogram data.

11. The information processing method according to claim 10, wherein
the predetermined condition further includes that the predetermined abnormality has not been found in any of a plurality of diagnoses based on the electrocardiogram data of the subject.

12. The information processing apparatus according to claim 1, wherein
the predetermined condition further includes that no congenital abnormality related to the predetermined abnormality has been found in the subject.

13. The information processing apparatus according to claim 1, wherein
the predetermined condition further includes that a blood relative of the subject has no medical history of a predetermined disease related to the predetermined abnormality.

14. The information processing apparatus according to claim 4, wherein
the predetermined condition includes that the predetermined abnormality has been found in a plurality of diagnoses for the subject.

15. The information processing apparatus according to claim 4, wherein
the predetermined condition includes that the predetermined abnormality has not been found in a diagnosis using the electrocardiogram data.

16. The information processing apparatus according to claim 4, wherein
the predetermined condition includes that no abnormality related to the predetermined abnormality has been found in a diagnosis using the electrocardiogram data.
